(19) United States
(12) Patent Application Publication
(10) Pub. No.: US 2026/0278891 A1
(43) Pub. Date: Sep. 17, 2026

(54) RECONSTRUCTING IMAGE DATA FROM MAGNETIC RESONANCE K-SPACE DATA

(71) Applicant: Siemens Healthineers AG, Forchheim (DE)

(72) Inventor: Marcel Dominik Nickel, Herzogenaurach (DE)

(21) Appl. No.: 19/567,137

(22) Filed: Mar. 14, 2026

(30) Foreign Application Priority Data
Mar. 14, 2025 (DE) 10 2025 109 890.5

(51) Int. Cl. G06T 12/20; A61B 5/055; G01R 33/56

(52) U.S. Cl. CPC G06T 12/20; A61B 5/055; G01R 33/5608; G06T 2211/424; G06T 2211/441

(57) ABSTRACT

A computer-implemented method for reconstructing image data from magnetic resonance k-space data using a trained machine learning model includes receiving acquired k-space data. The acquired k-space data have an irregular sampling pattern in k-space. In order to generate image data, an iterative machine-learning-based k-space-to-image reconstruction is performed on the acquired k-space data with multiple reconstruction steps including iterating between reconstruction steps of applying a conventional image reconstruction involving a parallel imaging model relating image data to k-space data, and applying the trained machine learning model to enhance image data. At least one of the conventional image reconstruction steps and/or at least one additional reconstruction step includes adapting data at the current iteration state to be more suitable for the trained machine learning model and/or to correspond more closely to training data the trained machine learning model has been trained with.

This application claims the benefit of German Patent Application No. DE 10 2025 109 890.5, filed on Mar. 14, 2025, which is hereby incorporated by reference in its entirety.

### BACKGROUND

The present embodiments relate to reconstructing image data from magnetic resonance k-space data and generating magnetic resonance image data.

Independent of the grammatical term usage, individuals with male, female, or other gender identities are included within the term.

Magnetic resonance (MR) imaging (MRI) plays an important role in clinical diagnosis. However, the acquisition of MRI data typically requires substantial amounts of time, which requires the patient to hold still for a corresponding amount of time and/or to find a solution for correcting motion artefacts. Trying to avoid motion artefacts by avoiding movement, such as via breath holding, may cause discomfort for the patient. Therefore, it is desirable to significantly accelerate acquisitions while keeping image impression and contrast. In order to accelerate acquisition, under-sampling of k-space data during image acquisition has been applied in multiple variants. For this purpose, advanced imaging techniques have been introduced to enable the acceleration of image acquisition while maintaining a sufficiently high diagnostic confidence.

For example, parallel imaging and compressed sensing (CS) are methods that are used for this purpose. In parallel imaging, multiple receiver coils as well as knowledge about their positioning and sensitivities are used to assist a spatial localization of the acquired magnetic resonance signal (MR signal) in order to be able to reduce the number of phase encoding steps during image acquisition. Known parallel imaging techniques are, for example, GRAPPA (“GeneRalized Autocalibrating Partial Parallel Acquisition”), CAIPIRINHA (Controlled Aliasing in Parallel Imaging Results in Higher Acceleration), or SENSE (“SENStivity Encoding”). Compressed sensing modifies the image acquisition by using incoherent acquisitions with irregular sampling pattern combined with a non-linear image reconstruction. Using incoherent acquisitions with irregular sampling pattern allows to even further accelerate image acquisition times compared to the uniform sampling used for parallel imaging. However, in compressed sensing, the obtained images may look more artificial, and this may be one reason why the impact of compressed sensing has been rather moderate and limited to few clinical applications.

A more recent approach is applying an image reconstruction based on deep learning. Deep learning-based image reconstruction allows to significantly accelerate acquisitions by under-sampling while keeping image impression and contrast. One recent approach of a deep learning-based image reconstruction is described in “Prospective Deployment of Deep Learning Reconstruction Facilitates Highly Accelerated Upper Abdominal MRI,” Brendel, Jan M. et al., Academic Radiology, Volume 31, Issue 12, 4965-4973. In this publication, the deep learning-based image reconstruction is implemented in a two-step approach using six iterations that alternate between a conventional parallel imaging reconstruction and image enhancement employing a deep learning model based on a neural network (e.g., on three-dimensional U-nets). For example, the conventional parallel imaging reconstruction receives k-space data, a parallel imaging model (e.g., in form of coil sensitivity maps), and a prior image estimate to calculate a new image and where the output is evaluated through a neural network. This may be expressed as

$${x}_{n + 1 / 2} = {arg min}_{x} ( {‖ Ax - y ‖}^{2} + \frac{1}{{λ}_{n}^{2}} {‖ x - {x}_{n} ‖}^{2} ) , \qquad [ 1 ]$$

$${x}_{n + 1} = {U}_{n} ( {x}_{n + 1 / 2} ) . \qquad [ 2 ]$$

where x_{n+1/2} is the reconstructed image of the parallel imaging step, A is a system operator (e.g., parallel imaging model) that connects k-space data and a reconstructed image, y are the acquired k-space data, λₙ is a regularization parameter that may also be referred to as step-size, and xₙ may be considered as a prior image estimate. A may include coil sensitivities of the MR system. Equation [1] without the regularization term corresponds to a standard SENSE parallel imaging reconstruction. The regularization term provides that the problem is not underconstrained. Uₙ denotes a neural network for image enhancement or to estimate the next prior image. The term of equation [1] generates an image x_{n+1/2} from the prior image, and Uₙ (equation [2]) generates the next prior image xₙ₊₁ from the output of equation 1, which is fed back into equation [1].

However, one disadvantage of the deep-learning based image reconstruction is that since the method is based on parallel imaging, the improved acquisition speed tends to be slower than with compressed sensing, such that the advantageous acquisition speed of compressed sensing cannot be fully exploited.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that allows to further improve on an existing image reconstruction and/or acquisition methods and, for example, allows particularly quick image acquisition while retaining good image quality is provided.

In the following, the solution according to the present embodiments is described with respect to the methods as well as with respect to the system, computer program, and storage medium. Features, advantages, or alternative embodiments herein may be assigned to the other objects and vice versa. In other words, embodiments for the systems may be improved with features described in the context of the respective methods. In this case, the functional features of the method are implemented by physical units of the system.

According to a first aspect of the present embodiments, a computer-implemented method for reconstructing image data from magnetic resonance k-space data using a trained machine learning model (e.g., trained deep-learning model) is provided. The method includes: (a) receiving acquired k-space data, where the acquired k-space data has an irregular sampling pattern in k-space; (b) in order to generate image data, performing an iterative machine-learning-based k-space-to-image reconstruction on the acquired k-space data with multiple reconstruction steps including (i) iterating between reconstruction steps of applying a conventional image reconstruction relating image data to k-space data, and applying the trained machine learning model to enhance image data, (ii) where at least one of the conventional image reconstruction steps and/or at least one additional reconstruction step includes adapting data at the current iteration state to be more suitable for the trained machine learning model and/or to correspond more closely to training data the trained machine learning model has been trained with.

One idea of the method according to the present embodiments is to allow for a connection between deep learning-accelerated applications that are typically not optimized and trained for irregular sampling pattern and using incoherent acquisitions with irregular sampling pattern. Using irregular sampling allows to reduce the acquisition time of the MR acquisition. Therefore, part of the idea of the present embodiments is to acquire k-space data with irregular sampling pattern and then use a trained machine learning model for reconstruction, to combine quick acquisition with improved reconstruction quality, allowing to keep image impression and contrast at a high standard. However, the applicant has found that training deep learning based models with irregular sampling pattern is difficult, leading to inferior results compared to training with regular sampling patterns, and corresponding models tend to generalize less well. For example, a behavior for high frequencies tends to be problematic and is to be checked carefully by human inspection. Surprisingly, it has been found that the data, based on the irregular sampling pattern, may be adapted to be more suitable for trained machine learning models. For example, this may be achieved by adapting the data to correspond more closely to training data the trained machine learning model has been trained with. For example, the machine learning model may be trained with data that is suitable for the machine learning model, such as more uniform data having a more regular sampling pattern, and then, during application, the acquired data with irregular sampling pattern may be adapted to come close to the training data (e.g., be more regular). In one embodiment, the machine learning model may thus be applied to profit from the better reconstruction results due to the model, while at the same time, the faster acquisition speed of irregular sampling may be used without leading to increased difficulties in training and applying the model.

In general, a trained machine learning model mimics cognitive functions that humans associate with other human minds. For example, by training based on training data, the machine learning model is able to adapt to new circumstances and to detect and extrapolate patterns. Another term for “trained machine learning model” is “trained function.” In general, parameters of a machine learning model may be adapted by training. For example, supervised training, semi-supervised training, unsupervised training, reinforcement learning, and/or active learning may be used. Further, representation learning (e.g., an alternative term is “feature learning”) may be used. For example, the parameters of the machine learning models may be adapted iteratively by several steps of training. For example, within the training, a certain cost function may be minimized. For example, within the training of a neural network, the backpropagation algorithm may be used. For example, a machine learning model may include a neural network, a support vector machine, a decision tree, and/or a Bayesian network, and/or the machine learning model may be based on k-means clustering, Q-learning, genetic algorithms, and/or association rules. For example, a neural network may be a deep neural network, a convolutional neural network, or a convolutional deep neural network. Further, a neural network may be an adversarial network, a deep adversarial network, and/or a generative adversarial network.

The trained machine learning model used according to the present embodiments may, for example, be a deep-learning model. A deep-learning model may include a neural network having multiple layers (e.g., having at least three layers). However, the neural network may also have significantly more than three layers (e.g., hundreds or even thousands of layers). Hence, the word deep refers to there being multiple layers. Alternative terms for “neural network” are “artificial neural network,” “artificial neural net,” or “neural net.” For example, a deep-learning model may include a (deep) convolutional neural network and/or may include transformers. A convolutional neural network is a neural network that uses a convolution operation instead of a general matrix multiplication in at least one of its layers (e.g., the “convolutional layer”). For example, a convolutional layer performs a dot product of one or more convolution kernels with the convolutional layer's input data/image, where the entries of the one or more convolution kernel are the parameters or weights that are adapted by training. For example, the Frobenius inner product and the ReLU activation function may be used. A convolutional neural network may include additional layers (e.g., pooling layers, fully connected layers, and normalization layers). By using convolutional neural networks, input images may be processed in a very efficient way, because a convolution operation based on different kernels may extract various image features, so that by adapting the weights of the convolution kernel, the relevant image features may be found during training. Further, based on the weight-sharing in the convolutional kernels, less parameters are to be trained, which prevents overfitting in the training phase and allows to have faster training or more layers in the network, improving the performance of the network.

According to the present embodiments, the machine learning model may, for example, include a U-net structure. Within the U-net structure, first, the input image data is down-sampled (e.g., decreasing the size of the image data and increasing the number of channels); afterwards, the input image data is up-sampled (e.g., increasing the size of the images and decreasing the number of channels) to generate a transformed image. For example, a U-net with an architecture and trained as described in “Prospective Deployment of Deep Learning Reconstruction Facilitates Highly Accelerated Upper Abdominal MRI,” Brendel, Jan M. et al., Academic Radiology, Volume 31, Issue 12, 4965-4973 may also be applied for the present embodiments. The basic concepts of a U-net architecture and its implementation are described in Ronneberger, O., Fischer, P., Brox, T., “U-Net: Convolutional Networks for Biomedical Image Segmentation,” Navab, N., Hornegger, J., Wells, W., Frangi, A. (eds) Medical Image Computing and Computer-Assisted Intervention, MICCAI 2015, Lecture Notes in Computer Science, vol 9351, Springer, Cham. It has even been found that the model does not necessarily have to be trained any different than a typical machine learning model of the state of the art trained for image reconstruction, since the adapting of the data may allow to use a model that is trained not specifically for the irregular sampling pattern. Accordingly, the method according to the present embodiments may be used by applying an already existing machine learning model, therefore avoiding the need for carrying out a training and for requiring corresponding training data. However, a machine learning model may also be trained specifically to be applied in the method of the present embodiments.

The acquired k-space data may also be denoted as acquired magnetic resonance imaging k-space data. Hence, the k-space data is data that has been acquired by a magnetic resonance imaging system. The acquired k-space data has an irregular sampling pattern in k-space. An irregular sampling pattern may be understood in contrast to a regular sampling pattern. Generally, an irregular sampling pattern in k-space provides that the distances between neighboring sampling points are not uniform but vary over a k-space grid. An irregular sampling pattern may be a randomized or pseudo-randomized sampling pattern. Hence, the sampling points may be chosen randomly or randomly with certain constraints. The irregular sampling pattern may also be denoted as incoherent sampling pattern. The irregular sampling pattern may have a variable sampling density. For example, the irregular sampling pattern may have a higher sampling density close to a central part of k-space and a lower sampling density at the outer parts (e.g., periphery) of k-space. More relevant information is contained in the center of k-space than at the outer parts of k-space. The irregular sampling pattern may be a combination of a regular sampling pattern and irregular sampling pattern. A regular sampling pattern is, for example, used in parallel imaging. A regular sampling pattern, for example, provides that the acquisition is sped up by decreasing the sampling (e.g., under-sampling), but the sampling pattern is uniform, such that the distances between neighboring sampling points are equal.

After receiving the acquired k-space data, an iterative machine-learning-based k-space-to-image reconstruction is performed. Therefore, it is iterated between applying the conventional image reconstruction and applying the trained machine learning model. The iteration may be based on the iteration as describe above with equations [1] and [2], modified by adapting the data as described under part (ii) of the method. For example, equation [2] may optionally be applied as written. For example, equation [1] may be adapted for this method. The iterations are, for example, performed multiple times (e.g., 2 to 20 times, 3 to 10 times, or 4 to 8 times). For example, each of the two iterated reconstruction steps may be performed a number of times, such as five times, while alternating between the two steps. Applying the conventional image reconstruction includes, for example, applying a parallel imaging model. The parallel imaging model may include coil sensitivity maps. The coil sensitivity maps may be derived from reference data. The coil sensitivity maps may be adapted to the regular sampling pattern. Applying the parallel imaging model may include using a prior image estimate to calculate a new image. Initially, the prior image may be set randomly or by setting it to predefined values, such as to all zero. During iteration, the prior image may, for example, correspond to the output of the machine learning model. Hence, the machine learning model may output an adapted image that is used as input for the conventional imaging reconstruction during the next iterative step.

The parallel imaging model, for example, includes a forward model relating the image to the acquired k-space data (e.g., with a linear relation). The forward model may also be denoted as system operator. The forward model may be described as a linear relation in the compressed form

$$y = A x ,$$

where x is the image, y is the acquired k-space data, and A is the forward model. The forward model may include coil sensitivity information (e.g., the coil sensitivity maps). Using the indices/for the receive channel, k and p for the k-space position, as well as m for spatial coordinates, the forward model may be described based on the sum

$${y}_{I , k} = {\sum }_{p , m} {P}_{k , p} {u}_{p , m} {C}_{I , m} {x}_{m} ,$$

$$where$$

$${P}_{k , p} = \{ \begin{matrix}1 , & k = p and k acquired \\ 0 , & else\end{matrix}$$

is the projection on the sampled acquisitions, u_{p,m} are coefficients of a Fourier transform, and C_{I,m} are the coil sensitivity maps. Thus, the forward model may be described by the form

$${A}_{I , k , m} = {\sum }_{p} {P}_{k , p} {u}_{p , m} {C}_{I , m} .$$

The conventional image reconstruction may include a regularization parameterized by a parameter to a prior image of the iteration (e.g., a Tikhonov regularization term including a Tikhonov regularization parameter). The regularization may regulate the currently generated image with respect to the prior image. The regularization parameter may correspond to as step size of the iteration step. For example, the regularization (e.g., the Tikhonov regularization term) may be of the form

$$\frac{1}{{λ}_{n}^{2}} {‖ x - {x}_{n} ‖}^{2} ,$$

with λₙ being the Tikhonov regularization parameter or step size. The trained machine learning model is, for example, trained to enhance the image output of the conventional image reconstruction. The output of the trained machine learning model is, for example, the next prior image. For each of the iterative steps of applying the trained machine learning model, a different trained sub-model may be applied.

At least one of the conventional image reconstruction steps and/or at least one additional reconstruction step includes adapting data at the current iteration state. For example, the data may be adapted via the additional reconstruction step prior to starting the actual iterative steps. For example, the received k-space data may be adapted before starting the iterations. Adapting the data may include supplementing the data and/or completing the data (e.g., by artificially generated data). Artificially generated data may be data that is calculated and/or made up (e.g. randomly made up or made up according to predetermined rules). Making up data according to predetermined rules may include setting the data values or some of the data values to predetermined values. Additionally or alternatively, the data may be adapted during at least one of the conventional image reconstruction steps, such as the first conventional image reconstruction step or all of the conventional image reconstruction steps or a partial number of the conventional image reconstruction steps. The data is adapted to be more suitable for the trained machine learning model and/or to correspond more closely to training data the trained machine learning model has been trained with. For example, it has been found that machine learning models tend to be better suited and may be trained more easily with a uniform sampling pattern. Hence, adapting the data to be more suitable may include adapting the data to have a more uniform sampling pattern. A uniform sampling pattern may be denoted as conventional sampling pattern. It has been found that with a uniform sampling pattern, optimized system operators (e.g., the operator A in equation [1] above) that have a better performance regarding de-aliasing may be used. Coil sensitivity maps may be independent of the sampling pattern and may therefore be used also in compressed sensing. However, as has been found, for conventional samplings, the coil sensitivity maps may be optimized/adapted better.

According to an embodiment, the trained machine learning model is trained end-to-end over the whole iterative process such that the iterative machine-learning-based k-space-to-image reconstruction has been simultaneously updated during training. For example, the iterative steps of the iterative reconstruction may be updated simultaneously during training. For example, during training, the weights of the machine learning model may be adapted together with at least one regularization parameter (e.g., Tikhonov regularization parameter). It has been found that training end-to-end may give particularly good results allowing a very good adaption of the parameters. Alternatively, the at least one regularization parameter may be set heuristically. For example, the weight of the regularization may be set via setting and/or adapting the regularization parameter.

Additionally or alternatively, the trained machine learning model is trained based on regular parallel imaging k-space data with uniform sampling pattern. For example, the trained machine learning model is trained with training data that includes regular parallel imaging k-space data with uniform sampling pattern. For example, the machine learning model may be trained with regular parallel imaging k-space data based on SENSE, GRAPPA, or CAIPIRINHA.

The machine learning model may be trained in a supervised manner. For each of the iterative steps of applying the trained machine learning model, a different trained sub-model may be trained. The sub-models may be trained end-to-end (e.g., the sub-models may be updated simultaneously during training). The machine learning model may, for example, include a three-dimensional U-net architecture and be trained as described in “Prospective Deployment of Deep Learning Reconstruction Facilitates Highly Accelerated Upper Abdominal MRI,” Brendel, Jan M. et al., Academic Radiology, Volume 31, Issue 12, 4965-4973. Hence, as described in this paper, for supervised training, for example, 5000 pairs may be used that are derived from about 500 fully sampled datasets. The datasets may be obtained from volunteers (e.g., in head, abdomen, and pelvis) using magnetic resonance imaging scanners, such as 1.5 Tesla and/or 3 Tesla scanners. As loss function, a L1 metric may be used. As optimizer Adam may be used. The obtained intermediate images may be interpolated using a deep-learning-based super-resolution algorithm. The algorithm may initially perform a two-fold up-sampling (e.g., in all spatial directions) and enforce data consistency on the acquired frequencies.

In one embodiment, a machine learning model may be used that is already trained may be used. Hence, an additional training may not be necessary. For example, a standardized machine learning model may be used that may also be used for parallel imaging k-space data with regular sampling pattern.

Features, advantages, or alternative embodiments herein defined with respect to the training may be assigned to the other objects and vice versa.

According to an embodiment, the at least one additional reconstruction step is performed before starting to iterate between the reconstruction steps such that the current iteration state is the state before starting the iteration. For example, the data that is adapted may be the acquired k-space data. Optionally, the iterative steps may also be adapted. For example, the step of applying a conventional image reconstruction involving a parallel imaging model may be adapted such that the adapted data is included in this step. The data (e.g., the acquired k-space data) may be adapted to have a more uniform sampling pattern (e.g., to be supplemented with additional data to be more uniform). More uniform is to be understood such that the data is closer to a regular sampling pattern. For example, the acquired k-space data may be supplemented with additional data, and the iterative steps may be adapted to be applied based on both the acquired k-space data and supplemented k-space data. For example, a term may be added (e.g., in the conventional image reconstruction), introducing the supplemented k-space data. In one embodiment, according to this embodiment, only little adjustments, if any, are to be done to the iterative process itself.

According to an embodiment, the trained machine learning model is trained based on regular parallel imaging k-space data with uniform sampling pattern, where the irregular sampling pattern of the acquired k-space data is irregular in that, compared to regular parallel imaging k-space data, data points are missing at non-uniform positions in k-space due to under-sampling. Adapting data at the current iteration state includes synthesizing k-space data for at least some of the missing data points and supplementing the acquired k-space data with the synthesized k-space data in order to generate k-space data that is more uniform and closer to the under-sampling of regular parallel imaging k-space data (e.g., essentially completely uniform and equivalent to the under-sampling of regular parallel imaging k-space data). Adapting data may, for example, be performed before starting to iterate between the reconstruction steps such that the current iteration state is the state before starting the iteration. It has been found that supplementing the acquired k-space data works well to provide a more regular sampling pattern that allows the machine learning model to be trained more easily. This embodiment allows for a particularly smooth connection between machine learning-accelerated applications (e.g., deep-learning-accelerated applications), with the application of compressed sensing using irregular/variable sampling pattern. The more uniform or essentially completely uniform sampling pattern corresponds, for example, to a parallel imaging sampling pattern. The irregular sampling pattern of the acquired k-space data may be a combination of a regular sampling pattern and, on top of the regular sampling pattern, an irregular sampling pattern. Supplementing the irregular sampling pattern may include filling the grid positions of the regular sampling pattern that are missing in the irregular sampling pattern with the synthesized k-space data.

According to an embodiment, at least one of the conventional image reconstruction steps and/or the at least one additional reconstruction step includes: performing a compressed sensing reconstruction on the acquired k-space data in order to generate a CS-reconstructed image data, the compressed sensing reconstruction including a parallel imaging component and an image regularization function; based on the CS-reconstructed image data and on the parallel imaging component, performing the step of adapting the data by generating additional synthesized k-space data supplementing the acquired k-space data to be more uniform and closer to the under-sampling of regular parallel imaging k-space data (e.g., essentially completely uniform and equivalent to the under-sampling of regular parallel imaging k-space data); and performing the iterative machine-learning-based k-space-to-image reconstruction using the acquired k-space data and the synthesized k-space data.

Performing the compressed sensing reconstruction on the acquired k-space data may, for example, be carried out before iterating between the reconstruction steps. The parallel imaging component may, for example, be based on SENSE, GRAPPA, and/or CAIPIRINHA. The parallel imaging component may include a parallel imaging model and/or a set of coil sensitivity maps. The image regularization function may be a regularization function that enforces prior knowledge onto the reconstructed image. In the context of the present embodiments, the image regularization function may also be denoted as compressed sensing regularization function. The regularization function may be scaled by a regularization strength parameter. The image regularization function may, for example, be based on total variation and/or be wavelet-based. For example, the image regularization function may be the L1-Norm of a wavelet decomposition or total variation. For example, for a given wavelet transformation W(x), an image regularization function R(x) may be

$${R}^{( wavelet )} ( x ) = {‖ W ( x ) ‖}_{1}$$

and for a two-dimensional image with spatial indices m, n, the isotropic total variation may be described by

$${R}^{( 2 D TV isotropic )} ( x ) = {\sum }_{m , n} \sqrt{{\vert {x}_{m + 1 , n} - {x}_{m , n} \vert}^{2} + {\vert {x}_{m , n + 1} - {x}_{m , n} \vert}^{2}} ,$$

while the non-isotropic total variation may be described by

$${R}^{( 2 D TV non - isotropic )} ( x ) = {\sum }_{m , n} \vert {x}_{m + 1 , n} - {x}_{m , n} \vert + \vert {x}_{m , n + 1} - {x}_{m , n} \vert .$$

Similarly, for three-dimensional images with indices m, n, o, this may be described by

$${R}^{( 3 D TV isotropic )} ( x ) = {\sum }_{m , n} \sqrt{{\vert {x}_{m + 1 , n , o} - {x}_{m , n , o} \vert}^{2} + {\vert {x}_{m , n + 1 , o} - {x}_{m , n , o} \vert}^{2} + {\vert {x}_{m , n , o + 1} - {x}_{m , n , o} \vert}^{2}} ,$$

$${R}^{( 3 D TV non - isotropic )} ( x ) = {\sum }_{m , n} \vert {x}_{m + 1 , n , o} - {x}_{m , n , o} \vert + \vert {x}_{m , n + 1 , o} - {x}_{m , n , o} \vert + \vert {x}_{m , n , o + 1} - {x}_{m , n , o} \vert .$$

Optionally, the performing of a compressed sensing reconstruction may include optimizing a cost function, the cost function including the parallel imaging component. The cost function may be of the type argmin. The cost function may, for example, be optimized (e.g., minimized) with respect to the real space image data. For example, the compressed sensing reconstruction for generating the CS-reconstructed image data x^{(CS)} may be of the form

$${x}^{( CS )} = {arg min}_{x} ( {‖ {A}^{( CS )} x - y ‖}^{2} + R ( x ) ) , \qquad [ 3 ]$$

where A^{(CS)} corresponds to the forward model of the parallel imaging model used in compressed sensing, and R(x) is the image regularization function. For example, k-space data may be synthesized based on the term

$${y}^{′} = {\overline{A}}^{( CS )} {x}^{( C S )} , \qquad [ 4 ]$$

where y′ is the synthesized k-space data, and Ā^{(CS)} is a modification of a forward model used in compressed sensing that is used to generate the synthesized k-space data. The modification of the forward model may be based on the forward model described above and herein. For example, for synthesizing the k-space data points, only the operator P_{k,p} is to be changed (e.g., since the acquired data is specified). The forward model Ā^{(CS)} that synthesizes the k-space data may thus be based on the equation

$${y}_{I , k} = {\sum }_{p , m} {u}_{p , m} {C}_{I , m} {x}_{m} .$$

Different coil sensitivity maps may C_{I,m} be used than for regular sampling. For example, \tilde{P}_{k,p} may be defined by

$${\tilde{P}}_{k , p} = \{ \begin{matrix}1 , & k = p and k - space data to be synthesized \\ 0 , & else\end{matrix} .$$

Optionally, the iteration steps of applying a conventional image reconstruction each include optimizing a cost function, the cost function including a parallel imaging reconstruction model and a mapping of the synthesized k-space data from the CS-reconstructed image. The cost function may be of the type argmin. The cost function may, for example, be optimized with respect to the reconstructed image. For example, with respect to equation [1] above, the k-space data y may simply be replaced by the supplemented k-space data z including both y and y′, such that equation [1] is replaced at least for some iteration steps by

$${x}_{n + 1 / 2} = {arg min}_{x} ( {‖ Ax - z ‖}^{2} + \frac{1}{{λ}_{n}^{2}} {‖ x - {x}_{n} ‖}^{2} ) , \qquad [ 5 ]$$

where x_{n+1/2} is the reconstructed image of the parallel imaging step, A is a forward model (e.g., system operator of the parallel imaging model) that connects k-space data and a reconstructed image, λₙ is a regularization parameter (e.g., Tikhonov regularization parameter) that may also be referred to as step-size, and xₙ may be considered as a prior image estimate. The step-sizes λₙ may depend on the iteration, and Uₙ(·) denotes the machine learning algorithm (e.g., a deep-learning algorithm, such as including a neural network, for image enhancement).

According to an embodiment, the step of generating additional synthesized k-space data is integrated into the iteration steps of applying the conventional image reconstruction. For example, the iterative step of applying a conventional image reconstruction may include calculating the additional synthesized k-space data (e.g., by including a term for synthesizing the synthesized k-space data). For example, for at least some of the iteration steps, equation [1] or equation [5] may include the term on the right side of equation [4], such that

$${x}_{n + 1 / 2} = {arg min}_{x} ( {‖ Ax - y ‖}^{2} + {‖ \overline{A} x - {\overline{A}}^{( C S )} {x}^{( C S )} ‖}^{2} + \frac{1}{{λ}_{n}^{2}} {‖ x - {x}_{n} ‖}^{2} ) , \qquad [ 6 ]$$

where Ā is the modification of the operator A that maps onto the synthesized samples, and similarly Ā^{(CS)} is the modification of A^{(CS)} Compared to equation [1], the term ∥Ax−y∥² is thus supplemented by the term ∥Āₓ−Ā^{(CS)}x^{(CS)}∥² so that both acquired k-space data and synthesized k-space data are used for the calculation. Hence, in this example, the acquired k-space data and the synthesized k-space data each have a separate term in the applied conventional image reconstruction. However, the overall application of a parallel imaging model, such as in equation [1], remains.

According to an embodiment, adapting data in at least one of the conventional image reconstruction steps includes optimizing a cost function. The cost function includes a compressed sensing reconstruction model applied on the acquired k-space data. The compressed sensing reconstruction includes a parallel imaging component and an image regularization function and is configured to generate CS-reconstructed image data. The cost function further includes a parallel imaging reconstruction model with a mapping of the synthesized k-space data from the CS-reconstructed image data. For example, the step of generating additional synthesized k-space data thus may be integrated into the iteration steps of applying the conventional image reconstruction via the mapping of the synthesized k-space data from the CS-reconstructed image data. The CS-reconstructed image data is, for example, generated from a prior image. The prior image is an initial image at first and then the respective output of the machine learning model during the iterating. For example, thus, the iteration steps of applying a conventional image reconstruction may each include optimizing the cost function. The compressed sensing reconstruction model and the parallel imaging reconstruction model may each be configured as individual terms of the cost function. For example, the cost function may be configured to be optimized (e.g., minimized) with respect to the real space image data in the parallel imaging model and the CS-reconstructed image data. The mapping of the synthesized k-space data from the CS-reconstructed image data may be based on applying the parallel imaging component of the compressed sensing reconstruction model in the parallel imaging reconstruction. For example, the CS-reconstructed image data z may be mapped to the synthesized k-space via a term such as

$${‖ \overline{A} x - {\overline{A}}^{( C S )} z ‖}^{2}$$

In the cost function. The compressed sensing reconstruction model may, for example, be applied in each of the conventional image imaging reconstruction steps. The compressed sensing reconstruction model may be scaled via a hyperparameter. The hyperparameter may optionally be adapted during training of the machine learning model. The hyperparameter allows a feedback and/or conditioning to the compressed sensing reconstruction. The hyperparameter may be adapted differently for each iterative step. This may allow a greater flexibility for improving the quality of the method. For example, the hyperparameter may give more weight to the compressed sensing reconstruction at the beginning of the iterative process. The hyperparameter may be set to be the same for each iteration step. The hyperparameter may be fixed and not adapted during training. For example, equation [1] may be replaced by

$${x}_{n + 1 / 2} = {arg min}_{x} {min}_{z} ( {‖ Ax - y ‖}^{2} + {‖ \overline{A} x - {\overline{A}}^{( C S )} z ‖}^{2} + \frac{1}{{λ}_{n}^{2}} {‖ x - {x}_{n} ‖}^{2} + α ( {‖ {A}^{( CS )} z - y ‖}^{2} + R ( z ) ) ) , \qquad [ 7 ]$$

with the hyperparameter α, the CS-reconstructed image data z, and the image regularization function R(z). Generally, the variables may be equivalent to variables of equation [6] described above that are represented by the same designation. In equation [7], the term

$${‖ Ax - y ‖}^{2} + {‖ \overline{A} x - {\overline{A}}^{( C S )} z ‖}^{2} + \frac{1}{{λ}_{n}^{2}} {‖ x - {x}_{n} ‖}^{2}$$

corresponds to the parallel imaging reconstruction model and the term

$$α ( {‖ {A}^{( C S )} z - y ‖}^{2} + R ( z ) )$$

corresponds to the compressed sensing reconstruction model. In one embodiment, the aspect of applying the compressed sensing reconstruction, the synthesizing and parallel imaging step of the machine learning based reconstruction may thus be combined in one iterative step. It has been found that such a combination may be advantageous so that, via the iteration process, the quality of the compressed sensing reconstruction itself may also benefit from the output of the machine learning model, since all the aspects are interconnected via the iterative process.

According to an embodiment, the compressed sensing reconstruction model includes a regularization parameterized by a parameter to a prior image of the iteration (e.g., a Tikhonov regularization term). The Tikhonov regularization term may be a scaled Tikhonov regularization term. The regularization may, for example, be scaled by a scaling parameter. Via the regularization (e.g., the Tikhonov regularization term), the output of the machine learning model may be fed into the compressed sensing reconstruction model (e.g., to regulate the compressed sensing reconstruction via the prior image output from the machine learning model). Hence, via the regularization, it may be provided that the CS-reconstructed image data is generated from a prior image. The prior image is an initial image at first and then the respective output of the machine learning model during the iterating. In one embodiment, the resulting reconstruction may thus be even better. This embodiment may, for example, be implemented by extending equation [7] such that the following equation is applied:

$${x}_{n + 1 / 2} = {arg min}_{x} {min}_{z} ( {‖ Ax - y ‖}^{2} + {‖ \overline{A} x - {\overline{A}}^{( C S )} z ‖}^{2} + \frac{1}{{λ}_{n}^{2}} {‖ x - {x}_{n} ‖}^{2} + α ( {‖ {A}^{( C S )} z - y ‖}^{2} + R ( z ) + \frac{β}{{λ}_{n}^{2}} {‖ z - {x}_{n} ‖}^{2} ) ) , \qquad [ 8 ]$$

$$with the regularization ,$$

$$\frac{β}{{λ}_{n}^{2}} {‖ z - {x}_{n} ‖}^{2}$$

being scaled by the scaling parameter β. The influence of the prior image on the compressed sensing reconstruction is scaled via the scaling parameter β. The scaling parameter may be fixed. For example, the scaling parameter may be set by a user. Alternatively, the scaling parameter may be adapted during training of the machine learning model. In one embodiment, via the regularization, the compressed sensing reconstruction may efficiently be conditioned by the prior image output by the machine learning model and the previous guess of image reconstruction while iterating.

According to an embodiment, a hull (e.g., a convex hull) is drawn in the phase encoding plane in k-space. Any synthesized k-space data outside the hull is synthesized by setting it to a predefined value (e.g., to essentially zero or to zero). In one embodiment, more relevant information is in the center area of k-space. Accordingly, setting the synthesized k-space data to zero may not disturb the reconstructed image. Further, setting the outer data points to zero will avoid high-frequency image artifacts that may otherwise arise.

According to an embodiment, the conventional image reconstruction step includes applying a parallel imaging reconstruction model and adapting data in the at least one of the conventional image reconstruction steps is realized by adding an image regularization function to the parallel imaging reconstruction model. This embodiment may provide a relatively simple way of adapting the data. The image regularization function may, for example, be added in addition to a regularization parameterized by a parameter to a prior image of the iteration (e.g., a Tikhonov regularization term). Hence, for example, equation [1] may be replaced by

$${x}_{n + 1 / 2} = {arg min}_{x} ( {‖ Ax - y ‖}^{2} + \frac{1}{{λ}_{n}^{2}} {‖ x - {x}_{n} ‖}^{2} + R ( x ) ) , \qquad [ 9 ]$$

with the image regularization function R(x), which is added to the parallel imaging reconstruction model in addition to the regularization

$$\frac{1}{{λ}_{n}^{2}} {‖ x - {x}_{n} ‖}^{2} .$$

According to an embodiment, the acquired irregular sampling pattern is a regular sampling pattern that is further modified by an irregular sampling pattern such that the acquired irregular sampling pattern is a combination of a regular sampling pattern and an irregular sampling pattern. A regular sampling pattern may be used in parallel imaging.

A regular sampling pattern, for example, provides that the acquisition is sped up by decreasing the sampling rate, but the sampling pattern is uniform, such that the distances between neighboring sampling points are essentially equal. Combining a regular sampling pattern and an irregular sampling pattern may be understood such that generally only grid points according to the regular sampling pattern are populated, but additionally, some of these regular grid points are also omitted leading to an even faster acquisition. Accordingly, a combination of regular sampling and irregular sampling leads to an irregular sampling. This combination may allow to accelerate acquisition significantly while also providing that the machine learning model may be trainable relatively easily and perform particularly good. This may, for example, be achieved by adapting the acquired data to correspond to the regular sampling pattern by filling the gaps in the irregular sampling grid. The regular sampling pattern and the irregular sampling pattern may be controlled by separate parameters. The parameters may control the density of the respective sampling and may be multiplied in order to combine regular sampling and irregular sampling. The parameters may, for example, describe a degree of sampling. For example, a parameter R may be a number describing a factor between the number of grid points of a sampled grid compared to the number of grid points of a full (e.g., non-under-sampled) grid. For example, with regular sampling, if both in vertical and horizontal direction, every second grid point is omitted a sampling factor of R=4 may be the parameter, providing the full grid has four times the grid points than the sampled grid.

According to a further aspect of the present embodiments, a method for generating magnetic resonance image data is provided. The method includes the acts of: acquiring k-space data with an irregular sampling pattern via a magnetic resonance imaging system; and performing a method as described herein on the acquiring k-space data.

All features and advantages of the method for reconstructing image data may be adapted to the method for generating magnetic resonance image data and vice versa.

According to an embodiment, the k-space data is acquired with the irregular sampling pattern such that a regular sampling pattern that is further modified by an irregular sampling pattern is acquired so that the acquired irregular sampling pattern is a combination of a regular sampling pattern and an irregular sampling pattern. The same advantages and modifications may apply as described above with respect to the combination of regular sampling pattern and an irregular sampling pattern.

According to a further aspect of the present embodiments, a computer program is provided including instructions that, when the program is executed by a computer, cause the computer to carry out any of the methods described herein. All features and advantages of the method for reconstructing image data and of the method for generating magnetic resonance image data may be adapted to the computer program and vice versa.

According to a further aspect of the present embodiments, a computer-readable storage medium (e.g., non-transient storage medium) is provided including instructions that, when executed by a computer, cause the computer to carry out any of the methods described herein. All features and advantages of the method for reconstructing image data, of the method for generating magnetic resonance image data, and of the computer program may be adapted to the computer-readable storage medium and vice versa. The storage medium may be any computer-readable storage medium. For example, the storage medium may be an optical storage medium or a solid-state storage medium. The storage medium may be a hard-drive, a solid-state-disk, a flash storage, an online server, etc.

According to a further aspect of the present embodiments, a magnetic resonance imaging system is provided. The magnetic resonance imaging system is configured to carry out any of the methods as described herein and/or includes a processing unit that is configured to carry out any of the methods as described herein. All features and advantages of the method for reconstructing image data, of the method for generating magnetic resonance image data, of the computer program, and of the computer-readable storage medium may be adapted to the magnetic resonance imaging system and vice versa.

The embodiments described herein may be combined with each other unless indicated otherwise.

### BRIEF DESCRIPTION OF THE DRAWINGS

The accompanying drawings illustrate various example embodiments and methods of various aspects of the invention.

FIG. 1 shows an example of a regular sampling pattern in k-space;

FIG. 2 shows an example of an irregular sampling pattern in k-space;

FIG. 3 shows an example of an irregular sampling pattern in k-space that is a combination of a regular sampling pattern as shown in FIG. 1 and an irregular sampling pattern;

FIG. 4 shows a diagram of a computer-implemented method for reconstructing image data from magnetic resonance k-space data according to an embodiment;

FIG. 5 shows a diagram of a computer-implemented method for reconstructing image data from magnetic resonance k-space data according to another embodiment;

FIG. 6 shows a diagram of a computer-implemented method for reconstructing image data from magnetic resonance k-space data according to another embodiment;

FIG. 7 shows a diagram of a computer-implemented method for reconstructing image data from magnetic resonance k-space data according to another embodiment;

FIG. 8 shows a diagram of a computer-implemented method generating magnetic resonance image data according to an embodiment; and

FIG. 9 shows a magnetic resonance imaging system according to an embodiment.

### DETAILED DESCRIPTION

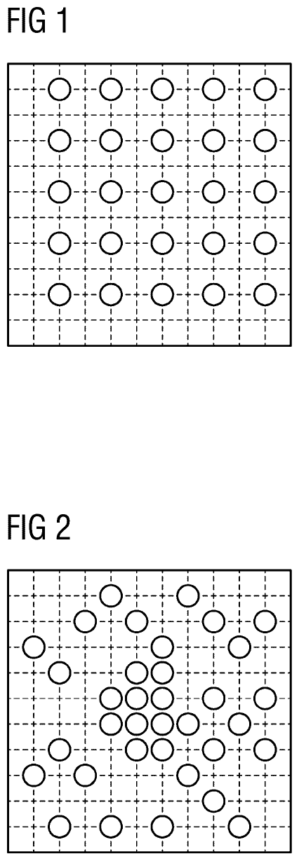

FIG. 1 shows an example of a regular sampling pattern in k-space. In this example, the regular sampling pattern corresponds to a regular under-sampling where in both vertical and horizontal direction every second position on the grid (e.g., marked by the dotted lines) is sampled. This leads to a sampling factor of R=4, providing that only one of every 4 grid positions are sampled. The sampled positions are marked by the large dots that sit on the grids, and these sampled positions are arranged equidistantly. The regular sampling has therefore a uniform density and samples all frequencies in the k-space that are shown here, including the high frequencies at the outer parts of k-space. This is a sampling that is typically used for parallel imaging such as GRAPPA.

FIG. 2 shows an example of an irregular sampling pattern in k-space. Hence, the sampled positions are arranged in a non-uniform manner, with varying distances between the sampled positions. The irregular sampling pattern has a variable density that, for example, focuses on lower resolution such that the sampling density is lower for the higher frequencies at the outer parts of the shown k-space. In this example, the irregular sampling pattern has a sampling factor of R=4 (e.g., the same sampling factor as the regular sampling in FIG. 1).

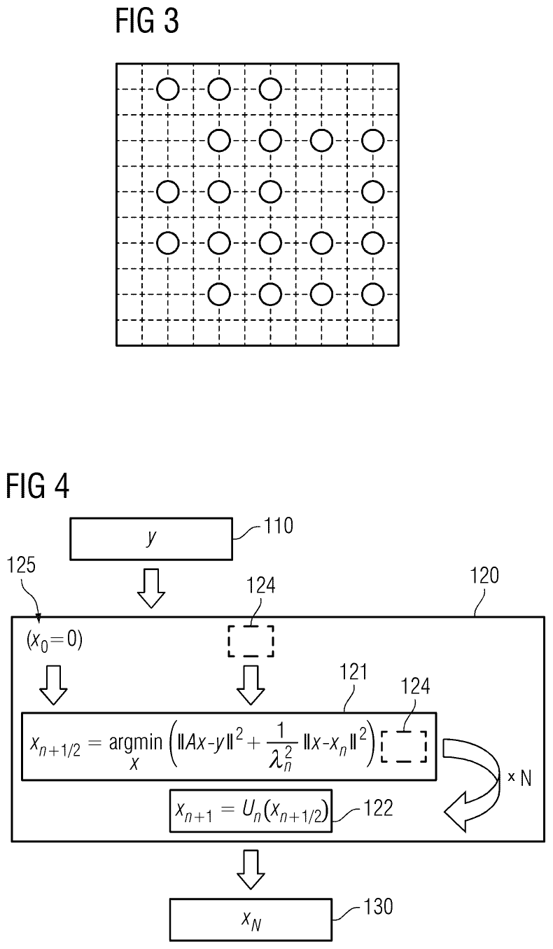

FIG. 3 shows an example of an irregular sampling pattern in k-space that is a combination of a regular sampling pattern as shown in FIG. 1 and an irregular sampling pattern. The regular sampling pattern of FIG. 1 is further modified by the irregular sampling pattern, such that the sampling factor R=5 is lower than the sampling factor in FIG. 1. Since this irregular sampling pattern is based on the regular sampling pattern in FIG. 1, only grid positions that are also populated in the regular sampling pattern of FIG. 1 are populated here. On top of this, some of the grid positions that are sampled in FIG. 1 are not sampled in FIG. 3, leading to an overall irregular sampling pattern, where the distances between sampled grid positions are not uniform.

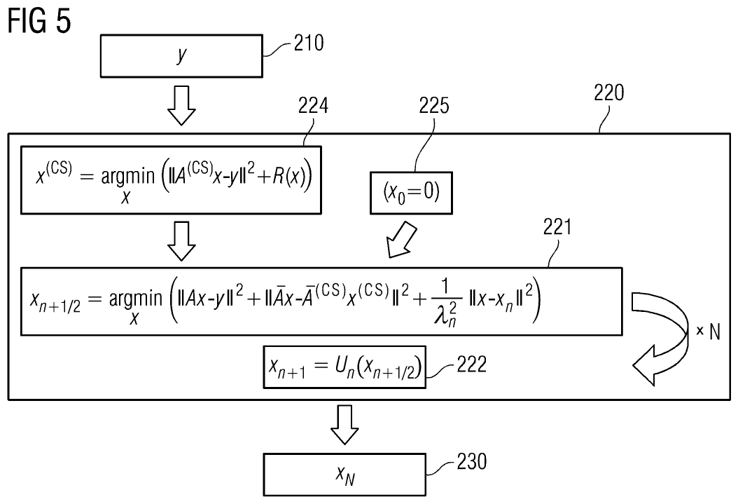

FIG. 4 shows a diagram of a computer-implemented method for reconstructing image data from magnetic resonance k-space data according to an embodiment. In a first act 110, acquired k-space data having an irregular sampling pattern in k-space is received. In a further act 120, an iterative machine-learning-based k-space-to-image reconstruction is performed on the acquired k-space data with multiple reconstruction steps. The reconstruction steps include iterating between a step 121 of applying a conventional image reconstruction involving a parallel imaging model relating image data to k-space data and a step 122 of applying a trained machine learning model (e.g., trained deep-learning model) to enhance image data. It is iterated multiple (N) times between these two steps. For example, both steps may be applied five or six times alternatingly. The conventional image reconstruction relates an image x to the k-space data y via a system operator A. The system operator A may include information about coil sensitivity maps. The coil sensitivity maps may, for example, be derived from reference data. The conventional image reconstruction also includes a regularization parametrized by the parameter λ to a prior image. Initially 125, in the first of the iteration steps 121, the prior image x₀ is given an initial value (e.g., it is set to zero). The iterative step 121 of the conventional image reconstruction thus generates an image x_{n+1/2} that is then, in the next iterative step 122, fed into the machine learning model that is trained to enhance the input image outputting another image xₙ₊₁ that, in turn, in the next iterative step 121, is again used as input for the conventional image reconstruction (e.g., as prior image (i.e., xₙ in the formula shown)). In addition to these steps, data at a current iteration state is adapted 124 to be more suitable for the trained machine learning model and/or to correspond more closely to training data the trained machine learning model has been trained with. This step 124 may, for example, be performed during at least one of the conventional image reconstruction steps 121 and/or before these steps 121. In a following act 130, when the iteration is finished, the generated image data x_{N} may be provided.

Optionally, in this or any of the other embodiments of the method, the generated image data x_{N} may be interpolated towards a target resolution (e.g., towards a higher resolution). For example, a deep-learning network including a super-resolution network may be applied on the generated image data x_{N} having as additional input a mask of acquired frequencies in Fourier domain. Accordingly, a final image with the target resolution may be generated.

FIG. 5 shows a diagram of a computer-implemented method for reconstructing image data from magnetic resonance k-space data according to another embodiment. In a first act 210, acquired k-space data having an irregular sampling pattern in k-space is received. The irregular sampling pattern of the acquired k-space data is irregular in that, compared to regular parallel imaging k-space data, data points are missing at non-uniform positions in k-space due to under-sampling. Hence, a similar irregular sampling pattern as described with respect to FIG. 3 may be used. In a further act 220, an iterative machine-learning-based k-space-to-image reconstruction is performed on the acquired k-space data with multiple reconstruction steps. Before starting the actual iteration, an additional reconstruction step 224 is applied in which a compressed sensing reconstruction on the acquired k-space data y is performed in order to generate CS-reconstructed image data x^{(CS)} The compressed sensing reconstruction includes a parallel imaging component ∥A^{(CS)}x−y∥² and an image regularization function R(x). After this, it is iterated between a step 221 of applying a conventional image reconstruction involving a parallel imaging model relating image data to k-space data and a step 222 of applying a trained machine learning model (e.g., trained deep-learning model) to enhance image data. It is iterated multiple (N) times between these two steps. The conventional image reconstruction relates an image x to the k-space data y via a system operator A. The system operator A may include information about coil sensitivity maps. During this iteration step 221 and the following iteration steps 221, k-space data for at least some of the missing data points are synthesized (e.g., via the term Ā^{(CS)}x^{(CS)}). Hence, in this embodiment, the step of generating additional synthesized k-space data is integrated into the iteration steps 221 of applying the conventional image imaging reconstruction. The synthesized k-space data are used to supplement the acquired k-space data (e.g., by adding the term ∥Āx−Ā^{(CS)}x^{(CS)}∥² that corresponds to the synthesized k-space data to the term ∥Ax−y∥² that corresponds to the acquired k-space data). Thus, essentially, data that is uniform and equivalent to the under-sampling of regular parallel imaging k-space data is generated, and the iterative machine-learning-based k-space-to-image reconstruction is performed using the acquired k-space data and the synthesized k-space data. The conventional image reconstruction step 221 also includes a regularization parametrized by the parameter A to a prior image. Initially 225, in the first of the iteration steps 221, the prior image x₀ is given an initial value (e.g., it is set to zero). The iterative step 221 of the conventional image reconstruction thus generates an image x_{n+1/2} that is then, in the next iterative step 222, fed into the machine learning model that is trained to enhance the input image and to output another image xₙ₊₁. The trained machine learning model is trained based on regular parallel imaging k-space data with uniform sampling pattern. Thus, by supplementing the acquired data with the synthesized data, the data is well-suited to be processed by the trained machine learning model. The output of the trained machine learning model step 222 is, in the next iterative step 221, again used as input for the conventional image reconstruction (e.g., as prior image (i.e., xₙ in the formula shown)). Both iterative steps 221, 222 are performed N times (e.g., six times) until, in a following act 230, when the iteration is finished, the generated image data x_{N} is provided.

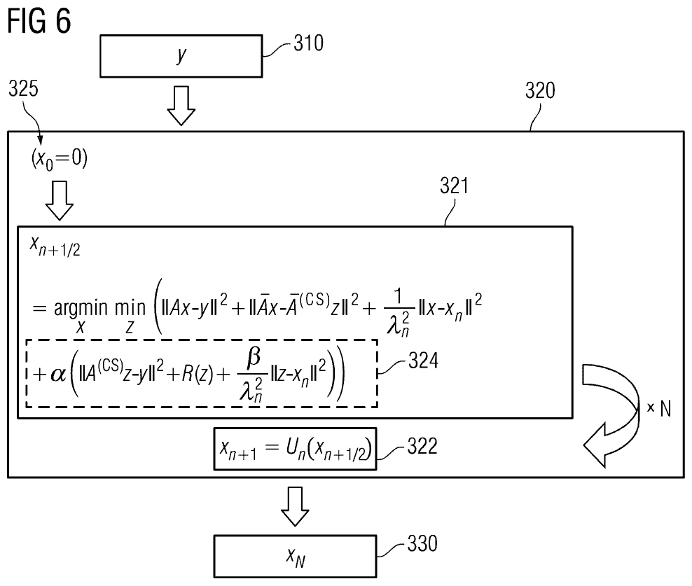

FIG. 6 shows a diagram of a computer-implemented method for reconstructing image data from magnetic resonance k-space data according to another embodiment. In a first act 310, acquired k-space data having an irregular sampling pattern in k-space is received. The irregular sampling pattern of the acquired k-space data is irregular in that, compared to regular parallel imaging k-space data, data points are missing at non-uniform positions in k-space due to under-sampling. In a further act 320, an iterative machine-learning-based k-space-to-image reconstruction is performed on the acquired k-space data with multiple reconstruction steps. Therein, it is iterated between a step 321 of applying a conventional image reconstruction involving a parallel imaging model relating image data to k-space data and a step 322 of applying a trained machine learning model (e.g., trained deep-learning model) to enhance image data. It is iterated multiple (N) times between these two steps. The conventional image reconstruction step includes optimizing a cost function (“argmin”). The cost function includes a compressed sensing reconstruction model 324 that is applied on the acquired k-space data y. The compressed sensing reconstruction includes a parallel imaging component ∥A^{(CS)}z−y∥², an image regularization function R(z), and a regularization

$$\frac{β}{{λ}_{n}^{2}} {‖ z - {x}_{n} ‖}^{2}$$

to a prior image of the iteration that is parameterized by a parameter λₙ, which is, for example, a scaled Tikhonov regularization term with a Tikhonov parameter. The compressed sensing reconstruction is configured to generate CS-reconstructed image data z. During this iteration step 321 and the following iteration steps 321, k-space data for at least some of the missing data points are synthesized (e.g., via the term Ā^{(CS)}z). Hence, in this embodiment, the step of generating additional synthesized k-space data is integrated into the iteration steps 321 of applying the conventional image imaging reconstruction. The synthesized k-space data are used to supplement the acquired k-space data (e.g., by adding the term ∥Āx−Ā^{(CS)}x^{(CS)}∥² that corresponds to the synthesized k-space data to the term ∥Ax−y∥² that corresponds to the acquired k-space data). Thus, essentially, data that is uniform and equivalent to the under-sampling of regular parallel imaging k-space data is generated, and the iterative machine-learning-based k-space-to-image reconstruction is performed using the acquired k-space data and the synthesized k-space data. Via a parallel imaging reconstruction mode, the conventional image reconstruction relates an image x to the k-space data y by using a system operator A of and to the synthesized k-space data z by using a system operator A. The system operator may include information about coil sensitivity maps. The conventional image reconstruction step 321 also includes a regularization

$$\frac{1}{{λ}_{n}^{2}} {‖ x - {x}_{n} ‖}^{2}$$

parametrized by the parameter λ to a prior image. Initially 325, in the first of the iteration steps 321, the prior image x₀ is given an initial value (e.g., it is set to zero). The iterative step 321 of the conventional image reconstruction thus generates an image x_{n+1/2} that is then, in the next iterative step 322, fed into the machine learning model, which is trained to enhance the input image and to output another image xₙ₊₁. The trained machine learning model is trained based on regular parallel imaging k-space data with uniform sampling pattern. Thus, by supplementing the acquired data with the synthesized data, the data is well-suited to be processed by the trained machine learning model. The output of the trained machine learning model step 322 is, in the next iterative step 321, used as input for the conventional image reconstruction (e.g., as prior image (i.e., xₙ in the formula shown)). Both iterative steps 321, 322 are performed N times (e.g., six times, until, in a following act 330, when the iteration is finished, the generated image data x_{N} is provided).

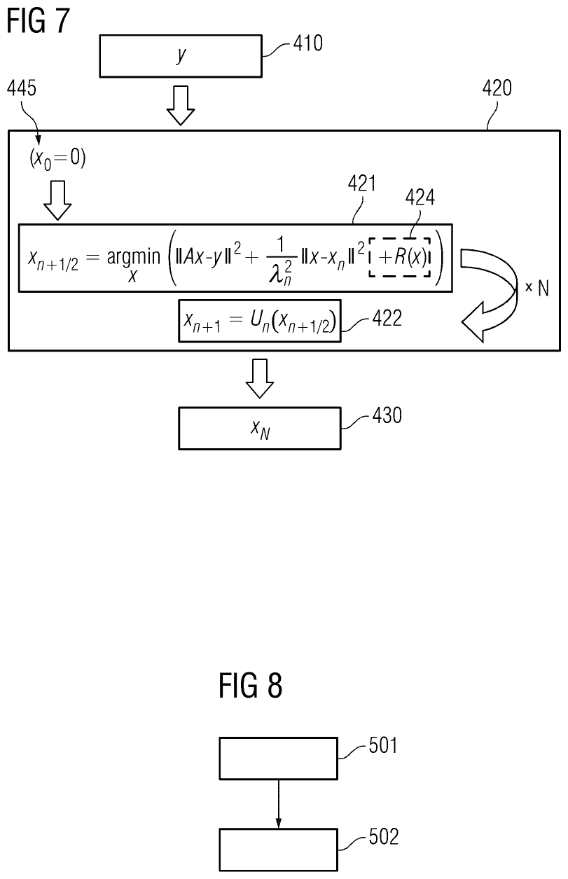

FIG. 7 shows a diagram of a computer-implemented method for reconstructing image data from magnetic resonance k-space data according to another embodiment. In a first act 410, acquired k-space data having an irregular sampling pattern in k-space is received. In a further act 420, an iterative machine-learning-based k-space-to-image reconstruction is performed on the acquired k-space data with multiple reconstruction steps. The reconstruction steps include iterating between a step 421 of applying a conventional image reconstruction involving a parallel imaging model relating image data to k-space data and a step 422 of applying a trained machine learning model (e.g., trained deep-learning model) to enhance image data. It is iterated multiple (N) times (e.g., five or six times) between these two steps. The conventional image reconstruction relates an image x to the k-space data y via a system operator A. The system operator A may include information about coil sensitivity maps. In addition, during these steps 421 of applying a conventional image reconstruction, data at a current iteration state is adapted 424 to be more suitable for the trained machine learning model via the inclusion of an additional image regularization function R(x) that is included in the parallel imaging reconstruction model.

The conventional image reconstruction also includes a regularization

$$\frac{1}{{λ}_{n}^{2}} {‖ x - {x}_{n} ‖}^{2}$$

parametrized by the parameter λ to a prior image. Initially 425, in the first of the iteration steps 421, the prior image x₀ is given an initial value (e.g., it is set to zero). The iterative step 421 of the conventional image reconstruction thus generates an image x_{n+1/2} that is then, in the next iterative step 422, fed into the machine learning model that is trained to enhance the input image outputting another image xₙ₊₁ that, in turn, in the next iterative step 421, is used as input for the conventional image reconstruction (e.g., as prior image (i.e., xₙ in the formula shown)). In a following act 430, when the iteration is finished, the generated image data x_{N} is provided.

FIG. 8 shows a diagram of a computer-implemented method generating magnetic resonance image data according to an embodiment. In a first act 510, k-space data is acquired with an irregular sampling pattern via a magnetic resonance imaging system. The irregular sampling pattern may be acquired such that a regular sampling pattern that is further modified by an irregular sampling pattern is acquired so that the acquired irregular sampling pattern is a combination of a regular sampling pattern and an irregular sampling pattern. In a further act 502, a method as described in any one of FIGS. 4 to 7 is performed on the acquired k-space data.

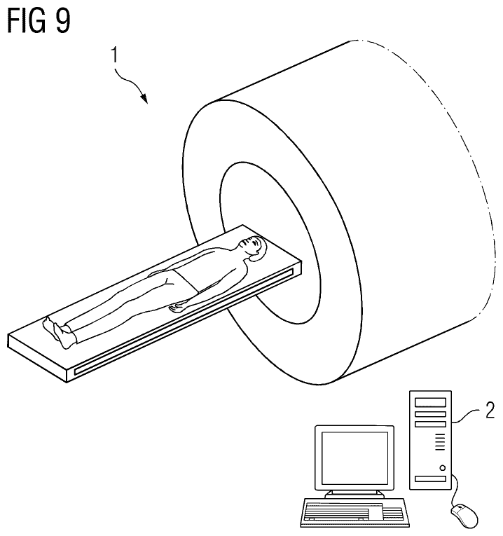

FIG. 9 shows a magnetic resonance imaging system 1 according to an embodiment including a processing unit 2 that is configured to carry out the method as described with respect to any one of the FIGS. 4 to 8.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for reconstructing image data from magnetic resonance k-space data using a trained machine learning model, the method being computer-implemented and comprising:
receiving acquired k-space data, wherein the acquired k-space data has an irregular sampling pattern in k-space; and
in order to generate image data, performing an iterative machine-learning-based k-space-to-image reconstruction on the acquired k-space data with multiple reconstruction steps, the multiple reconstruction steps comprising:
iterating between reconstruction steps of:
applying a conventional image reconstruction involving a parallel imaging model relating image data to k-space data; and
applying the trained machine learning model to enhance image data,
wherein at least one of the conventional image reconstruction steps, at least one additional reconstruction step, or the at least one conventional image reconstruction step and the at least one additional reconstruction step comprise adapting data at a current iteration state to be more suitable for the trained machine learning model, to correspond more closely to training data the trained machine learning model has been trained with, or a combination thereof.

2. The method of claim 1, wherein the trained machine learning model is a trained deep-learning model.

3. The method of claim 1, wherein the at least one additional reconstruction step is performed before starting to iterate between the reconstruction steps, such that the current iteration state is a state before starting the iteration.

4. The method of claim 1, wherein the trained machine learning model is trained based on regular parallel imaging k-space data with uniform sampling pattern,
wherein the irregular sampling pattern of the acquired k-space data is irregular in that, compared to regular parallel imaging k-space data, data points are missing at non-uniform positions in k-space due to under-sampling, and
wherein adapting data at the current iteration state comprises synthesizing k-space data for at least some of the missing data points and supplementing the acquired k-space data with the synthesized k-space data in order to generate k-space data that is more uniform and closer to the under-sampling of regular parallel imaging k-space data.

5. The method of claim 4, wherein adapting data at the current iteration state comprises synthesizing k-space data for at least some of the missing data points and supplementing the acquired k-space data with the synthesized k-space data in order to generate k-space data that is essentially completely uniform and equivalent to the under-sampling of regular parallel imaging k-space data.

6. The method of claim 1, wherein at least one of the conventional image reconstruction steps, the at least one additional reconstruction step, or the at least one conventional image reconstruction step and the at least one additional reconstruction step comprise:
performing a compressed sensing reconstruction on the acquired k-space data in order to generate CS-reconstructed image data, the compressed sensing reconstruction comprising a parallel imaging component and an image regularization function;
based on the CS-reconstructed image data and based on the parallel imaging component, adapting the data, the adapting of the data comprising generating additional synthesized k-space data supplementing the acquired k-space data to be more uniform and closer to the under-sampling of regular parallel imaging k-space data; and
performing the iterative machine-learning-based k-space-to-image reconstruction using the acquired k-space data and the synthesized k-space data.

7. The method of claim 6, wherein generating the additional synthesized k-space data supplementing the acquired k-space data to be more uniform and closer to the under-sampling of regular parallel imaging k-space data comprises generating the additional synthesized k-space data supplementing the acquired k-space data to be essentially completely uniform and equivalent to the under-sampling of regular parallel imaging k-space data.

8. The method of claim 6, wherein the generating of the additional synthesized k-space data is integrated into the iteration steps of applying the conventional image imaging reconstruction.

9. The method of claim 8, wherein the iteration steps of applying a conventional image imaging reconstruction each comprise optimizing a cost function, the cost function comprising a parallel imaging reconstruction model and a mapping of the synthesized k-space data from the CS-reconstructed image.

10. The method of claim 1, wherein adapting data in at least one of the conventional image imaging reconstruction steps comprises optimizing a cost function, the cost function comprising:
a compressed sensing reconstruction model applied on the acquired k-space data, the compressed sensing reconstruction comprising a parallel imaging component and an image regularization function, and being configured to generate CS-reconstructed image data; and
a parallel imaging reconstruction model with a mapping of the synthesized k-space data from the CS-reconstructed image data.

11. The method of claim 10, wherein the compressed sensing reconstruction model comprises a regularization parameterized by a parameter to a prior image of the iteration.

12. The method of claim 11, wherein the regularization parameterized by the parameter to the prior image of the iteration includes a Tikhonov regularization term with a Tikhonov parameter.

13. The method of claim 4, wherein a hull is drawn in the phase encoding plane in k-space, and
wherein any synthesized k-space data outside the hull is synthesized by setting the k-space data outside the hull to a predefined value.

14. The method of claim 13, wherein the hull is a convex hull.

15. The method of claim 1, wherein the conventional image reconstruction step comprises applying a parallel imaging reconstruction model and adapting data in the at least one of the conventional image reconstruction steps is realized at least by adding an image regularization function to the parallel imaging reconstruction model.

16. The method of claim 1, wherein:
the trained machine learning model is trained end-to-end over the whole iterative process, such that the iterative machine-learning-based k-space-to-image reconstruction has been simultaneously updated during training;
the trained machine learning model is trained based on regular parallel imaging k-space data with uniform sampling pattern; or
a combination thereof.

17. A method for generating magnetic resonance image data, the method comprising:
acquiring k-space data with an irregular sampling pattern via a magnetic resonance imaging system; and
reconstructing image data using the acquired k-space data, using a trained machine learning model, the reconstructing being computer-implemented and comprising:
receiving the acquired k-space data, wherein the acquired k-space data has an irregular sampling pattern in k-space; and
in order to generate image data, performing an iterative machine-learning-based k-space-to-image reconstruction on the acquired k-space data with multiple reconstruction steps, the multiple reconstruction steps comprising:
iterating between reconstruction steps of:
applying a conventional image reconstruction involving a parallel imaging model relating image data to k-space data; and
applying the trained machine learning model to enhance image data,
wherein at least one of the conventional image reconstruction steps, at least one additional reconstruction step, or the at least one conventional image reconstruction step and the at least one additional reconstruction step comprise adapting data at a current iteration state to be more suitable for the trained machine learning model, to correspond more closely to training data the trained machine learning model has been trained with, or a combination thereof.

18. The method of claim 12, wherein the k-space data is acquired such that with the irregular sampling pattern, a regular sampling pattern that is further modified by an irregular sampling pattern is acquired so that the acquired irregular sampling pattern is a combination of a regular sampling pattern and an irregular sampling pattern.

19. In a non-transitory computer-readable storage medium that stores instructions executable by a computer to reconstruct image data from magnetic resonance k-space data using a trained machine learning model, the instructions comprising:
receiving acquired k-space data, wherein the acquired k-space data has an irregular sampling pattern in k-space; and
in order to generate image data, performing an iterative machine-learning-based k-space-to-image reconstruction on the acquired k-space data with multiple reconstruction steps, the multiple reconstruction steps comprising:
iterating between reconstruction steps of:
applying a conventional image reconstruction involving a parallel imaging model relating image data to k-space data; and
applying the trained machine learning model to enhance image data,
wherein at least one of the conventional image reconstruction steps, at least one additional reconstruction step, or the at least one conventional image reconstruction step and the at least one additional reconstruction step comprise adapting data at a current iteration state to be more suitable for the trained machine learning model, to correspond more closely to training data the trained machine learning model has been trained with, or a combination thereof.

20. A magnetic resonance imaging system comprising:
a processing unit configured to reconstruct image data from magnetic resonance k-space data using a trained machine learning model, the processing unit being configured to reconstruct the image data comprising the processing unit being configured to:
receive acquired k-space data, wherein the acquired k-space data has an irregular sampling pattern in k-space; and
in order to generate image data, perform an iterative machine-learning-based k-space-to-image reconstruction on the acquired k-space data with multiple reconstruction steps, the multiple reconstruction steps comprising:
iteration between reconstruction steps of:
application of a conventional image reconstruction involving a parallel imaging model relating image data to k-space data; and
application of the trained machine learning model to enhance image data,
wherein at least one of the conventional image reconstruction steps, at least one additional reconstruction step, or the at least one conventional image reconstruction step and the at least one additional reconstruction step comprise adaptation of data at a current iteration state to be more suitable for the trained machine learning model, to correspond more closely to training data the trained machine learning model has been trained with, or a combination thereof.
